(19) United States
(12) Patent Application Publication
(10) Pub. No.: US 2022/0378322 A1
(43) Pub. Date: Dec. 1, 2022

(54) BREATHING COACH APPLICATION

(71) Applicant: Kashtin COOK, Grande Prairie (CA)

(72) Inventor: Kashtin COOK, Grande Prairie (CA)

(21) Appl. No.: 17/876,744

(22) Filed: Jul. 29, 2022

Related U.S. Application Data
(63) Continuation-in-part of application No. 17/334,891, filed on May 31, 2021.

(51) Int. Cl. A61B 5/08; A61B 5/024; A61B 5/0205; A61B 5/00

(52) U.S. Cl. CPC A61B 5/0816; A61B 5/024; A61B 5/02055; A61B 5/681; A61B 5/7455; A61B 5/7405

(57) ABSTRACT

A breathing coach system and method is disclosed comprising a biometric device capable of measuring one or more biometric measurements of a user; an application executed on a processor on a wearable device monitoring said one or more biometric measurements coupled to said biometric device and notifying the user to perform a recommended breathing pattern to effect said one or more biometric measurement when one or more of said one or more biometric measurements is within a predetermined region.

### BACKGROUND

Smart watches (or other smart biometric devices) have the ability to monitor the respiration rate of the user. This measurement is typically used to provide statistics after the fact. There is a need to use these measurements to provide instant feedback to a user to alter their breathing pattern to help with anxiety and relaxation. By taking control of their breathing in real time, the user can impact their well-being all-day long.

### BRIEF SUMMARY

In a first embodiment, a breathing coach system and method is disclosed comprising a biometric device capable of measuring one or more biometric measurements of a user; an application executed on a processor on a wearable device monitoring said one or more biometric measurements coupled to said biometric device and notifying the user to perform a recommended breathing pattern to effect said one or more biometric measurement when one or more of said one or more biometric measurements is within a predetermined region.

In another embodiment, said one or more biometric measurements comprises a heart rate, a blood pressure or body temperature.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a wearable smart device in accordance with one embodiment.

FIG. 2 illustrates an embodiment of the breathing coach system.

### DETAILED DESCRIPTION

The details of one or more embodiments of the subject matter of this specification are set forth in the accompanying drawings and the description below. Other features, aspects, and advantages of the subject matter will become apparent from the description, the drawings, and the claims.

Like reference numbers and designations in the various drawings indicate like elements.

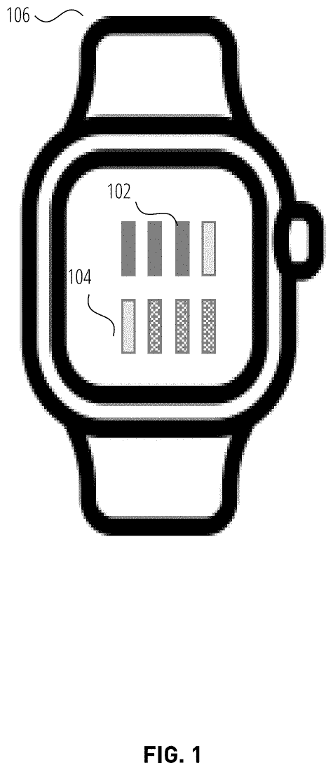

FIG. 1 depicts a wearable smart device 106 (for example, but not limited to a smart watch) capable of measuring respiration rate or receiving respiration rate measurements from a biometric device. An application downloaded on the wearable smart device 106 displays bars reflecting the inhale level 102 and the exhale level 104 of the users as measured and compared to a baseline natural conscious breathing of the user.

In another embodiment, the biometric device is a separate device coupled with the application device via a communication protocol.

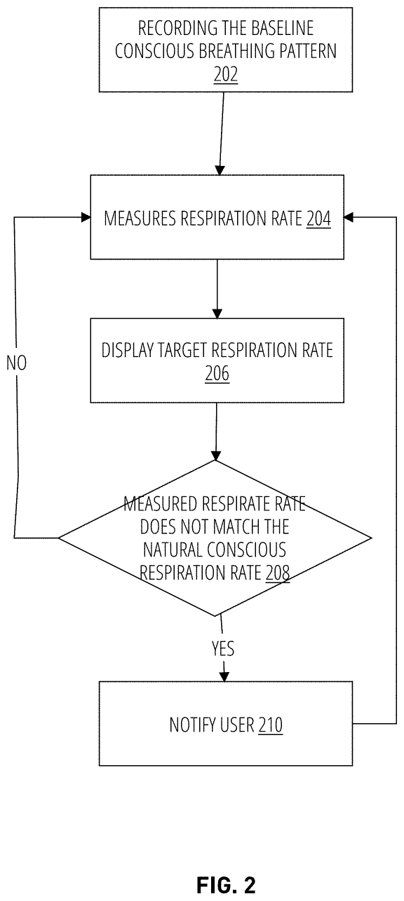

As per FIG. 2, the user initializes the application by recording the baseline conscious breathing pattern 202. The conscious breathing pattern is stored in the wearable smart device 106 as the baseline.

Then the application measures respiration rate 204 and displays target respiration rate 206 by showing the inhale level 102 and exhale level 104 on the screen of the wearable smart device 106. The levels and speed can be shown using bars as shown in FIG. 1. For example, the inhale level 102 is shown with bars lighting up from left to right and the exhale level 104 with the bars going from right to left. If the application is configured to have a pause between the inhale and the exhale, and/or the exhale and the inhale, then another graphic icon can be used as a counter for the pause.

The display could be of any designs that allow to show the two dimension of the respiration, the rate and the depth.

If the measured respiration rate does not match the baseline 208, the user is notified 210 using a vibration or a sound generated by the wearable smart device 106. For example if the user tries to exhale before the 4th bar of the inhale is reached.

The user can configure the application to make different sounds, types of vibrations and levels thereof.

The application measures respiration rates 204 on an ongoing basis and continuously compares to the baseline to provide continuous feedback. Optionally, the application can be configured via setting to only provide feedback during specific times of day.

As the user goes on about its day and forgets to breath consciously, the watch will constantly remind the user to go back to their baseline natural conscious breathing pattern.

In another embodiment, the application can be configured to implement different types of breathing rhythms (or predetermined breathing patterns) including a pause after the inhale and/or before the exhale. Optionally, the application can use different types of vibration for the inhale, pause, exhale. For example, but not limited to, the 4,4,4 box breathing technic used navy seals. Any other types of breathing techniques or patterns can be programmed into the application.

Using the application, the user does not need to worry about counting and they can focus on the breath and visualize toxic energy leaving their body. A subtle vibration of the wearable smart device 106 on the wrist triggers the user to inhale and hold their breath for 4 seconds, then another vibration will notify the user to exhale. The application acts as a metronome for the breathing of the user causing ongoing real-time relaxation.

In another embodiment, the application on the wearable smart device 106 will cause the user to take deeper breaths than the baseline to increase relaxation. Using this setting the vibration may optionally feel different or a different sound can be played. In this embodiment, the application acts as a metronome for breathing.

In another embodiment, the application records the breathing pattern of the user. Using this data, the application can display the breathing pattern in comparison with previous time segments or in comparison with the baseline conscious breathing pattern to show improvements achieved using the notifications.

In another embodiment, the application provides statistics of the number of time the notifications are sent, these statistics can show the user improvement over time.

In another embodiment, the application uses the heart rate measured by the device 106 to determine whether to execute a predetermined breathing pattern is needed. When the measured heart rate is within a predetermined region (e.g. higher than a first threshold and lower than a second threshold), a breathing pattern is recommended using sound and/or vibration notification on the wearable device. Optionally, multiple predetermined regions can be established, and a different breathing pattern may be used when the heart rate enters a different region. If the user's heart rate lowers to another pre-determined region, the application resets to a normal breathing pattern and stops the notifications.

For example, if the user's heart rate is above 120 bpm and below 150 bpm a first breathing pattern is executed on the wearable device 106. If the heart rate exceeds 150 bpm, a second breathing pattern is executed. When the heart rate falls below 120 bpm, then the breathing notifications stop.

In another embodiment, the application uses the blood pressure measured by the device to calculate which type of predetermined breathing pattern is needed. When the measured blood pressure is within a predetermined region, a breathing pattern is recommended using sound or vibration notification on the wearable device. If the user follows the breathing pattern and the blood pressure lowers to another pre-determined region, the application reset to a normal breathing pattern and stops the notifications.

In another embodiment, before the user takes a blood pressure test the device, the user notifies the application and a predetermined breathing pattern is executed for a predetermined time (e.g. 60 seconds) to lower the blood pressure. For example, the breathing pattern would be a 60 second slower and deep breathing rhythm, vibrating at the end of each inhale and exhale signaling the user to breath the opposite direction, for 60 seconds or until the resting heart rate is below a threshold (e.g. 60 bpm) before the user starts the blood pressure test.

In another embodiment, the application uses the body temperature measured by the device to calculate which type of predetermined breathing pattern is needed. When the measured temperature is within a predetermined region, a breathing pattern is recommended using sound or vibration notification on the wearable device. If the user follows the breathing pattern and the temperature changes to another pre-determined region, the application resets to a normal breathing pattern and stops the notifications.

In another embodiment, the application uses the respiration rate measured by the device to calculate which type of predetermined breathing pattern is needed. When the measured respiration rate is within a predetermined region, a breathing pattern is recommended using sound or vibration notification on the wearable device. If the user follows the breathing pattern and the respiration rate changes to another pre-determined region, the application reset to a normal breathing pattern and stops the notifications.

In another embodiment, the biometric device uses a pulse oximeter, and the respiration rate is inferred from the oxygen saturation.

The recommended breathing pattern may include a combination of longer, deeper, shallower, shorter breath.

Optionally, the application takes into account the activity of the user. The user enters the activity it is doing from a predetermined list (e.g. weight training, running, cycling, working, meditating) using the application interface or voice commands. The application recommends a breathing pattern that is specific to the activity.

As a first example, the application helps a user to lower the heart rate. A user is exercising in a gym, with a heart rate at 170 bpm. However, the user could be preforming the same at 155 beats per minute. The application detects the high heart rate and selects a breathing pattern to help lower the heart rate. The user starts following the recommended breathing pattern (e.g. by taking deeper breaths) and the heart rate lowers.

The breathing application implements the necessary pattern which has a deeper breathing pattern uploaded right into the device 106, the rate, the speed, the depth is all predetermined by the application with the goal to lower the heart rate.

As another example, the application helps lowering high heart rate while relaxing. A user is running the breathing application on their wearable device 106 while relaxing but the resting heart rate is at 75 bmp. The heart rate could be a bit lower given the level of activity they are doing. The application sets a breathing pattern to a specific rate, depth and speed to bring the heart rate down to a set goal of 60 beats per minute. When the set goal of 60 beats per minute is reached the deeper slower breathing pattern reverts to the original predetermined breathing pattern. The original breathing pattern being what the user is uses by default with the application.

As another example, the user sets the application to “exercising” and the heart rate is at 120 beats per minute which is within the predetermined target but the breathing is faster and shallower than the set goals. The application selects and executes a pre-determined breathing pattern that triggers the user to take deeper inhales and exhales with at lower speed between inhale and exhale. The user follows the recommended breathing pattern to get more oxygen and further lowers the hearth rate.

In another embodiment, the application includes voice recognition or a sensor that picks up the voice vibrations thru the wrist. With this feature, the breathing notifications are silences when the user talks, as the user does not breath normally when talking, which would trigger a lot of notifications. Once the user stops talking for a predetermined amount of time, the silent mode is turned off and the user starts to receive breathing notifications again, as described above.

The user of the application sets up several parameters such as age, height, weight, gender, other biometric parameters such as resting heart rate along with predetermined thresholds on the rate and depth that will trigger notifications. Predetermined breathing patterns may be downloaded customized to the user's specific information.

The foregoing descriptions of specific embodiments of the present invention have been presented for purposes of illustration and description. They are not intended to be exhaustive or to limit the invention and method of use to the precise forms disclosed.

For example, the vibration generated by the wearable device can be done by the face and/or wristband, in the case of a watch.

Obviously, many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient but is intended to cover the application or implementation without departing from the spirit or scope of the claims of the present invention.

What is claimed is:

1. A breathing coach system comprising:
a biometric device capable of measuring one or more biometric measurements of a user;
an application executed on a processor on a wearable device monitoring said one or more biometric measurements coupled to said biometric device and notifying the user to perform a recommended breathing pattern to effect said one or more biometric measurement when one or more of said one or more biometric measurements is within a predetermined region.

2. The system of claim 1 wherein said one or more biometric measurements comprises a heart rate.

3. The system of claim 1 wherein said one or more biometric measurements comprises a blood pressure.

4. The system of claim 1 wherein said one or more biometric measurements comprises a body temperature.

5. The system of claim 1 wherein said notifying is done by a vibration on the wearable device.

6. The system of claim 1 wherein said notifying is done by playing a tone on the smart device.

7. A method for coaching a user's breathing rate comprising:
measuring one or more biometric measurement of the user with a biometric device;
monitoring said one or more biometric measurements with an application coupled to said biometric device; and
notifying the user to perform a recommended breathing pattern to effect said one or more biometric measurement when one or more of said one or more biometric measurements is within a predetermined region

8. The method of claim 7 wherein said one or more biometric measurements comprises a heart rate.

9. The method of claim 7 wherein said one or more biometric measurements comprises a blood pressure.

10. The method of claim 7 wherein said one or more biometric measurements comprises a body temperature.

11. The method of claim 7 wherein said notifying is done by a vibration on the wearable device.

12. The system of claim 1 wherein said notifying is done by playing a tone on the smart device.
